(19) United States
(12) Patent Application Publication
(10) Pub. No.: US 2026/0365966 A1
(43) Pub. Date: Nov. 19, 2026

(54) METHOD FOR PRODUCING AN EXTRACT OF AMANITA GENUS FUNGI

(71) Applicant: Ievgen Vitaliiovych MAKEIEV, Dnipro (UA)

(72) Inventors: Ievgen Vitaliiovych MAKEIEV, Dnipro (UA); Viktor Mykolaiovych ANISHCHENKO, Kyiv (UA); Pavlo Oleksandrovych KUZEMA, Irpin (UA)

(21) Appl. No.: 19/189,327

(22) Filed: Apr. 25, 2025

(51) Int. Cl. C12N 1/14; A23L 33/10; A61K 36/06; C12R 1/645

(52) U.S. Cl. CPC C12N 1/14; A23L 33/10; A61K 36/06; C12R 2001/645

(57) ABSTRACT

The invention relates to the pharmaceutical and food industries, in particular to a method for producing an extract of Amanita genus fungi, which can later be used in the production of drugs and dietary supplements.

A method for producing an extract of Amanita genus fungi has been developed, which comprises: obtaining dried biomass of Amanita genus fungi; performing aqueous or aqueous-alcoholic extraction of the dried biomass of said fungi to produce an initial extract; separating solid particles of the biomass of said fungi from the initial extract; wherein, after separating solid particles of the biomass from the initial extract, a decarboxylation reaction of ibotenic acid in said extract is carried out to produce the final extract of Amanita genus fungi by adding to the initial extract, from which solid particles have been separated, a compound of structural formula (III):

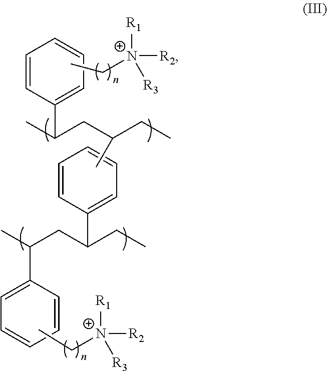

where R₁, R₂, R₃, k and n are as defined in the description of the invention, and furthermore an extract of Amanita genus fungi has been produced by the above-mentioned method.

The invention relates to the pharmaceutical and food industries, in particular to a method for producing an extract of Amanita genus fungi, which can later be used in the production of drugs and dietary supplements.

Fly agaric (Amanita) represents a genus of mycorrhizal, lamellar fungi belonging to the Amanita family (Amanitaceae), which comprises approximately 600 species of various fungi. These fungi are found in deciduous, coniferous, and mixed forests throughout North America, Siberia, and Europe.

Amanita genus fungi, including Amanita muscaria, Amanita pantherina, are widely used as a medicinal raw material in traditional medicine as well as in homeopathy. The medicinal properties of said fungi have long been used to treat the diseases of joints and back (rheumatism, arthritis, arthrosis, osteochondrosis, radiculitis), skin (ulcers, including cancerous ones, fistulas, bedsores, boils and carbuncles, dermatitis, eczema, fungal infections of the skin and nails, etc.), varicose veins and thrombophlebitis, allergies, benign tumours (myomas, fibromyomas, papillomas, warts, etc.) ( [symbol] H. [symbol] . [symbol] [symbol] [symbol] [symbol] [symbol] / [symbol] H. [symbol] ., [symbol] O. M., [symbol] B. B.// [symbol] [symbol] [symbol] : [symbol] Ta [symbol] : [symbol] [symbol] [symbol] , 3 [symbol] [symbol] [symbol] , 10 [symbol] 2021 p./M-BO [symbol] [symbol] , [symbol] [symbol] [symbol] - [symbol] , 2021.-c. 244). In addition, the indigenous peoples of Chukotka, Kamchatka, and Alaska used fly agaric to treat nervous and mental disorders, boost vitality, and relieve physical fatigue, and folk medicines containing fly agarics were also used to treat diseases of internal organs, tuberculosis, multiple sclerosis, functional disorders of the spinal cord, and gout ( [symbol] A. [symbol] - [symbol] [symbol] [symbol] [symbol] / [symbol] [symbol] - [symbol] - [symbol] : [symbol] , 2005.-63 c.).

It is also known, for example, that Amanita muscaria was used in homeopathy to prepare the Agaricus muscarius medication, which was used to treat vascular spasms, epileptic and choreatic conditions, multiple sclerosis, and functional disorders of the spinal cord ( [symbol] , 1958). Amanita genus fungi are well known for their—effects on the nervous system. In particular, the following species of Amanita genus fungi such as Amanita muscaria, Amanita pantherina, Amanita citrina, Amanita tomentella, Amanita porphyria, and Amanita regalis can affect the nervous system, causing psychotomimetic symptoms and, in some cases, hallucinations.

All the above-mentioned properties of Amanita genus fungi are determined by the composition of the chemicals that they contain. The fruit bodies of said fungi contain a variety of biologically active substances with concentrations that may vary depending on the species of fungi. The composition of the fungal fruit body includes, among others, acetylcholine, B vitamins (thiamine, riboflavin, pyridoxine, pantothenic and nicotinic acids, nicotinamide, folic acid and cobalamin), biotin, phylloquinone, ibotenic acid, muscimol, muscazone, muscarine, and other biologically active substances. Notably, these fungi contain compounds, such as ibotenic acid, muscimol, muscazone, and muscarine, that exhibit psychoactive effects. While muscazone and muscarine occur in very small amounts in the fruit bodies of, for example, Amanita muscaria, the total content of ibotenic acid and muscimol in this fungus can reach 0.18% by weight in the dried fungus. Moreover, the amounts of the above-mentioned substances can vary between different fungi. For example, in the Amanita pantherina fungus, the content of ibotenic acid is 0.46%, and muscimol is 0.2% by weight of the fresh fungus, while the amounts of muscazone and muscarine are minor (Benedict R. G., Tyler V. E., Brady L. R. Chemotaxonomic significance of isoxasole derivatives in Amanita species//Lloydia.-1966.-V.29.-p. 333-342). Thus, considering the total weight of said substances in these fungi, it can be assumed that ibotenic acid and muscimol are the main psychoactive substances in Amanita genus fungi, which can have significant effects on the human body.

Ibotenic acid, which has the structural formula (I):

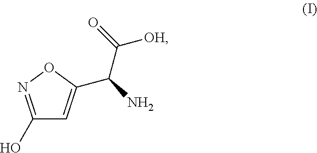 [SMILES: N[C@H](C(=O)O)c1cc(O)no1]

is a non-selective agonist of glutamate NMDA receptors, activation of which causes excitation of nerve cells and an increase in the Ca²⁺ level in them (Ibotenic acid mediates neurotoxicity and phosphoinositide hydrolysis by independent receptor mechanisms/Zinkand W. C., Moore W. C., Thompson C. [et al]//Mol. Chem. Neuropathol.-1992.-V.16, N21-2.-p. 1-10). Activation of NMDA receptors leads to the production of nitric oxide (NO) by Ca-dependent neuronal NO-synthase, and the release of nitric oxide triggers cell death. The ability of ibotenic acid to destroy brain tissue and cause neuronal death at the site of administration is frequently used in experimental research. Studies of brain sections have shown that the use of the extracts of Amanita muscaria and Amanita pantherina fungi causes activation of NMDA receptors in hippocampal neurons and simultaneously suppresses glutamate transmission. This suppression in certain brain structures plays an important role in the development of mental disorders due to ibotenic acid poisoning. In addition, among other signs of intoxication are lacrimation and salivation, abdominal pain, indigestion, pupillary constriction, bradycardia, convulsions, as well as hearing and auditory hallucinations. Notably, ibotenic acid is a metabolic precursor of muscimol, and in natural conditions or during drying of fungi, ibotenic acid undergoes a decarboxylation process, converting into muscimol. However, only about 10-20% of this acid converts into muscimol after the decarboxylation process in natural conditions.

Muscimol, which has the structural formula (11):

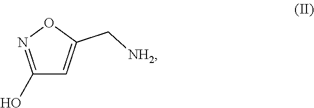

is a selective agonist of GABA_{A} receptors, which are the main inhibitory receptors in the central nervous system (CN S) (Johnston G. A. Muscimol as an ionotropic GA BA receptor agonist//Neurochemical Research.-2014.-V.39, N210.-p.1942-1947).

Due to the activation of GABA_{A} receptors, muscimol increases the flow of chloride ions into neurons, which leads to a decrease in the membrane potential of the cells (hyperpolarization), as well as a decrease in the level of their excessive excitation, which underlies excitotoxicity. Meanwhile, excessive glutamate release is a key factor in excitotoxicity. Muscimol indirectly suppresses this release, reducing the likelihood of neuronal damage resulting from excessive calcium influx through NMDA receptors. This substance therefore can potentially protect neurons from excitotoxic damage. Meanwhile, it should be noted that, due to suppression of excessive excitation, muscimol can potentially protect against ischaemic brain damage (for example, after a stroke), and due to suppression of excessive activation of glutamate pathways, it can potentially be used to treat conditions and diseases such as epilepsy, Alzheimer's disease, Huntington's disease and other neurodegenerative diseases. At the same time, muscimol can also cause a negative effect on the body, which manifests in a wide range of psychoactive effects, such as hallucination, euphoria, altered perception of time and space, drowsiness and sedative effect, that depend on dose and individual characteristics of the body. Furthermore, it can cause nausea, which can lead to vomiting, involuntary muscle contractions and tremor, impaired coordination of movements, and feelings of anxiety and fear.

Thus, due to the pharmacological effect of muscimol on the body, extracts from Amanita genus fungi with a high yield of said substance can potentially be used in the pharmaceutical industry, as well as in nutrition as a biologically active additive.

However, to produce the above-mentioned extract, it is necessary to use technological processes that will allow producing an extract in which the content of ibotenic acid will be minimized, or an extract completely devoid of this acid in its composition, since ibotenic acid is much more dangerous than muscimol and can cause ibotenate-induced seizures and lesions in specific regions of the brain, resembling those seen in Alzheimer's disease.

A method for producing an extract of Amanita muscaria fungi, which is described in the application US2024165180, is known from the art and, according to the inventors, is the closest analogue of the claimed method. The essence of this method is that aqueous extraction of dried Amanita muscaria fungi is initially carried out at a temperature ranging from 65 °C to 95 °C, then, upon completion of the extraction, the extract is filtered to remove fungal particles, afterwards 1 M hydrochloric acid is added to the filtered extract, lowering the pH value from 7 to 3 and initiating the process of decarboxylation of ibotenic acid to muscimol, and the produced mixture is further concentrated. According to the authors of the claimed invention, the disadvantage of this method is that the final extract still contains a relatively high amount of ibotenic acid.

The object of the invention is to develop a method for producing an extract of Amanita genus fungi, the implementation of which will ensure the achievement of a technical result consisting in producing an extract with a high yield of muscimol, in which the content of ibotenic acid will be minimized.

This object is achieved by developing a method for producing an extract of Amanita genus fungi, which comprises:
obtaining dried biomass of Amanita genus fungi;
performing aqueous or aqueous-alcoholic extraction of the dried biomass of said fungi to produce an initial extract;
separating solid particles of the biomass of said fungi from the initial extract;
wherein, after separating solid particles of the biomass from the initial extract, a decarboxylation reaction of ibotenic acid in said extract is carried out to produce the final extract of Amanita genus fungi by adding to the initial extract, from which solid particles have been separated, a compound of structural formula (III):

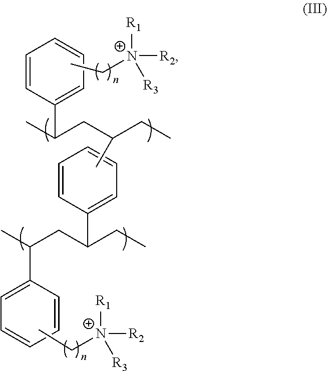

wherein
R₁ is CH₃;
R₂ is selected from the group comprising H and CH₃;
R₃ is selected from the group comprising H, CH₃, (CH₂)ₖCH₃, (CH₂)ₖOH, C₆H₄OH;
k is an integer from 1 to 17;
n is an integer from 1 to 10;
wherein if R₂ is H, R₃ is H,
and if R₂ is CH₃, R₃ is selected from the group comprising CH₃, (CH₂)ₖCH₃, (CH₂)ₖOH, C₆H₄OH.

A Iso, the set object is achieved by producing an extract of Amanita genus fungi using a method, comprising:
obtaining dried biomass of Amanita genus fungi;
performing aqueous or aqueous-alcoholic extraction of the dried biomass of said fungi to produce an initial extract;
separating solid particles of the biomass of said fungi from the initial extract;
wherein, after separating solid particles of the biomass from the initial extract, a decarboxylation reaction of ibotenic acid in said extract is carried out to produce the final extract of Amanita genus fungi by adding to the initial extract, from which solid particles have been separated, a compound of structural formula (III):

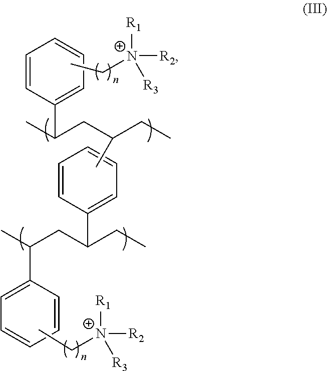

wherein
R₁ is CH₃;
R₂ is selected from the group comprising H and CH₃;
R₃ is selected from the group comprising H, CH₃, (CH₂)ₖCH₃, (CH₂)ₖOH, C₆H₄OH;
k is an integer from 1 to 17;
n is an integer from 1 to 10;
wherein if R₂ is H, R₃ is H,
and if R₂ is CH₃, R₃ is selected from the group comprising CH₃, (CH₂)ₖCH₃, (CH₂)ₖOH, C₆H₄OH.

The compound of structural formula (III) is a cross-linked polymer with grafted primary, and/or secondary, and/or tertiary, and/or quaternary organic amino groups, which is insoluble in water and in an aqueous-alcoholic mixture. It accelerates the decarboxylation reaction of ibotenic acid to muscimol by linking the hydrophilic cationic group of the compound of structural formula (III) to the carboxylate anion of ibotenic acid.

In this invention, compounds 1-6 of structural formula (III) can be used as catalysts in the decarboxylation reaction:

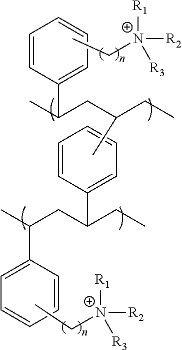

| Compound No. | R₁ | R₂ | R₃ |
|---|---|---|---|
| Compound 1 | CH₃ | H | H |
| Compound 2 | CH₃ | CH₃ | H |
| Compound 3 | CH₃ | CH₃ | CH₃ |
| Compound 4 | CH₃ | CH₃ | (CH₂)ₖCH₃ |
| Compound 5 | CH₃ | CH₃ | (CH₂)ₖOH |
| Compound 6 | CH₃ | CH₃ | C₆H₄OH. |

n = 1 - 10;
k= 1 - 17;

The process of decarboxylation of ibotenic acid to muscimol, induced by the compound of structural formula (III), is shown in Scheme 1.

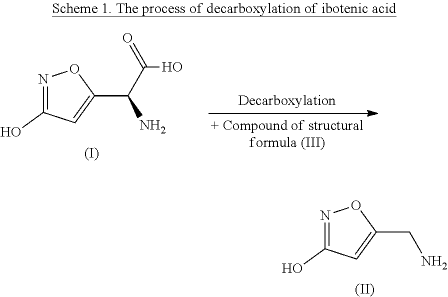

The aqueous or aqueous-alcoholic extraction of the biomass of Amanita genus fungi is preferably carried out in an inert atmosphere or air atmosphere at normal or reduced pressure.

Meanwhile, it should be noted that an air atmosphere at reduced pressure (<0.2 bar) or an inert atmosphere can further increase the concentration of ibotenic acid and muscimol in extracts compared to the extraction in an air atmosphere at normal pressure (1 bar).

Preferably, an organic or inorganic acid in an amount of 0.1-10 wt. % is added to the initial extract after adding the compound of structural formula (III). Adding acid to the extract reduces the pH value of the medium. Meanwhile, increased acidity of the medium facilitates the conversion of ibotenic acid into muscimol through the decarboxylation reaction. However, it should be noted that adding the acid to the extract under heterogeneous catalysis conditions is an optional step of the claimed method.

Most preferably, the organic acid is selected from the group comprising citric acid, acetic acid, formic acid, sorbic acid.

Also, the inorganic acid is most preferably selected from the group comprising hydrochloric acid and/or orthophosphoric acid.

In a preferred embodiment, Amanita genus fungi are selected from the group comprising Amanita muscaria, Amanita pantherina, Amanita citrina, Amanita tomentella, Amanita porphyria, Amanita regalis, Amanita albocreata, Amanita aliena, Amanita altipes, Amanita aprica, Amanita armeniaca, Amanita augusta, Amanita breckonii, Amanita chrysoblema, Amanita eliae, Amanita farinosa, Amanita frostiana, Amanita gemmata, Amanita gioiosa, Amanita ibotengutake, Amanita multisquamosa, Amanita nehuta, Amanita orientigemmata, Amanita parcivolvata, Amanita parvipantherina, Amanita persicina, Amanita petalinivolva, Amanita pseudoregalis, Amanita roseotincta, Amanita rubrovolvata, Amanita velatipes, Amanita virgineoides, Amanita viscidolutea, Amanita wellsii, Amanita xanthocephala, Amanita xylinivolva.

Meanwhile, it should be noted that all known species of Amanita genus fungi may be used in the claimed method, except for the Amanita phalloides, Amanita virosa, Amanita verna, Amanita bisporigera, Amanita ocreata species, which is due to the fact that they contain a poisonous α-amanitin compound.

In another preferred embodiment, either unground or ground biomass is used as the dried biomass of Amanita genus fungi.

Preferably, the dried biomass of Amanita genus fungi with a residual moisture content of 0.1-20% is used.

Fungi can be dried in a dehydrator or by applying heat to the fungi in any possible way known to those skilled in the art, including, but not limited to, conduction, convection or irradiation.

Preferably, the decarboxylation reaction of ibotenic acid in the initial extract is carried out at room temperature or within a temperature range of 30-95 °C for 2-48 hours.

Meanwhile, the pH of the initial extract during treatment with the compound of formula (III) can be in the range of 2-8.

In a preferred embodiment, the aqueous or aqueous-alcoholic extraction of the dried fungal biomass is carried out at a temperature of 5-40 °C for 0.5-10 hours.

In another preferred embodiment, the compound of formula (III) is solid and insoluble in water and in an aqueous-alcoholic mixture.

It should be noted that, after the completion of the extraction process under heterogeneous catalysis conditions, the content of ibotenic acid in the filtrate following its treatment is less than 0.1 μg/ml. Also, the above-mentioned extraction conditions make it possible to reduce the content of compounds with negatively charged groups in the solution, which increases the purity of muscimol in the final product.

In a preferred embodiment, after completing the decarboxylation reaction of ibotenic acid in the initial extract, the compound of structural formula (III) is separated from the final extract by filtration.

Meanwhile, filtration is carried out through a series of membranes with a pore size ranging from 0.22 μm to 100 μm, which makes it easy to separate even small catalyst particles. In addition, heterogeneous catalysis carried out using a solid catalyst has an advantage over the variant of homogeneous catalysis, which can also be used with corresponding organic amines. In the proposed variant of heterogeneous catalysis, a significant economic effect is achieved due to the possibility to use repeatedly a solid catalyst after an inexpensive procedure for its regeneration.

In a more preferred embodiment, the extraction and decarboxylation reaction of ibotenic acid in the initial extract are carried out in a sun-protected container.

These containers are necessary to avoid exposure to light throughout the extraction and decarboxylation process due to the possibility of muscimol decomposition.

Preferably, the separation of solid particles of the biomass from the initial extract is performed by centrifugation or filtration.

The solution produced after centrifugation or filtration can be subjected to additional filtration, if required, with or without applying external pressure. Meanwhile, filtration is carried out through a series of membranes with a pore size ranging from 0.22 μm to 130 μm for more thorough filtration of solid fungal particles.

Even more preferably, after completing the decarboxylation reaction of ibotenic acid in the initial extract, the final extract is purified to remove contaminants. Meanwhile, purification of the final extract can be performed using the methods of preparative chromatography, namely thin-layer chromatography, flash chromatography, ion-exchange chromatography.

Preferably, after completing the decarboxylation reaction of ibotenic acid in the initial extract, the final extract is either concentrated or dried.

In a preferred embodiment, concentrating the final extract involves distillation or solvent evaporation.

In this case, the final product is an aqueous or aqueous-alcoholic extract of Amanita fungi.

In another preferred embodiment, drying the final extract involves vacuum drying or lyophilization.

In this case, the final product is a dry extract of Amanita fungi.

In a preferred embodiment, the extract of Amanita genus fungi is used as a dietary supplement, formulated as a tablet, capsule, powder, pill, granule, solution, suspension, emulsion, tincture, gel, cream, ointment, or chewing gum.

In another preferred embodiment, the extract of Amanita genus fungi is used as a pharmaceutical composition, formulated as a tablet, capsule, powder, pill, granule, solution, suspension, emulsion, tincture, gel, cream, or ointment.

Meanwhile, said extracts of Amanita genus fungi are suitable for oral, sublingual, buccal, injectable, intranasal, inhalation or topical use, for example, but not limited to, for reduction of pain sensation, improvement of physical health and well-being, including relaxation and improved sleep.

Below are examples illustrating the implementation of the claimed method.

### EXAMPLE 1

Dry fruit bodies of Amanita fungi were sorted to remove off-grade specimens. The selected raw materials, without grinding, were poured with water at a rate of 150 ml per 10 g. The system consisting of liquid and solid material was stirred vigorously for 6 hours. After the extraction was completed, the produced solution was separated from solid particles using a centrifuge and subsequent filtration. The results of HPLC analysis showed that the muscimol content in the produced extract was 67 mg/I. A catalyst, which was a compound 4, was added to the filtrate at a rate of 50 g/l. The produced mixture was heated to 60 °C under constant stirring and kept at this temperature for 3 hours. After 3 hours, the mixture was cooled. Then it was analyzed using the HPLC method. The results of the analysis showed that the concentration of ibotenic acid was below the detection limit, and the concentration of muscimol was 105 μg/ml.

### EXAMPLE 2

Dry fruit bodies of Amanita fungi were sorted to remove off-grade specimens. The selected raw materials were ground to a fraction of less than 5 mm and poured with water at a rate of 150 ml per 10 g. The system consisting of liquid and solid material was stirred vigorously for 6 hours. After the extraction was completed, the produced solution was separated from solid particles using a centrifuge and subsequent filtration. The results of HPLC analysis showed that the muscimol content in the produced extract was 68 mg/I. A catalyst, which was a compound 5, was added to the filtrate at a rate of 50 g/l. The produced mixture was heated to 60 °C under constant stirring and kept at this temperature for 3 hours. After 3 hours, the mixture was cooled. Then it was analyzed using the HPLC method. The results of the analysis showed that the concentration of ibotenic acid was below the detection limit, and the concentration of muscimol was 107 μg/ml.

### EXAMPLE 3

Dry fruit bodies of Amanita fungi were sorted to remove off-grade specimens. The selected raw materials, without grinding, were poured with water at a rate of 150 ml per 10 g. The system consisting of liquid and solid material was stirred vigorously for 6 hours at a reduced pressure (0.2 atm). After the extraction was completed, the produced solution was separated from solid particles using a centrifuge and subsequent filtration. The results of HPLC analysis showed that the muscimol content in the produced extract was 74 mg/I. A catalyst, which was a compound 4, was added to the filtrate at a rate of 50 g/l. The produced mixture was heated to 60 °C under constant stirring and kept at this temperature for 3 hours. After 3 hours, the mixture was cooled. Then it was analyzed using the H PLC method. The results of the analysis showed that the concentration of ibotenic acid was below the detection limit, and the concentration of muscimol was 112 μg/ml.

### EXAMPLE 4

Dry fruit bodies of Amanita fungi were sorted to remove off-grade specimens. The selected raw materials, without grinding, were poured with water at a rate of 150 ml per 10 g. The system consisting of liquid and solid material was stirred vigorously for 6 hours. After the extraction was completed, the produced solution was separated from solid particles using a centrifuge and subsequent filtration. The results of HPLC analysis showed that the muscimol content in the produced extract was 67 mg/I. A catalyst, which was a compound 6, was added to the filtrate at a rate of 50 g/l. The produced mixture was heated to 60 °C under constant stirring and kept at this temperature for 5 hours. After 5 hours, the mixture was cooled. Then it was analyzed using the HPLC method. The results of the analysis showed that the concentration of ibotenic acid was below the detection limit, and the concentration of muscimol was 105 μg/ml.

### EXAMPLE 5

Dry fruit bodies of Amanita fungi were sorted to remove off-grade specimens. The selected raw materials, without grinding, were poured with water at a rate of 100 ml per 10 g. The system consisting of liquid and solid material was stirred vigorously for 10 hours. After the extraction was completed, the produced solution was separated from solid particles using a centrifuge and subsequent filtration. A catalyst, which was a compound 3, at a rate of 50 g/I, as well as citric acid at a concentration of 10 g/I were added to the filtrate. The produced mixture was heated to 70 °C under constant stirring and kept at this temperature for 3 hours. After 3 hours, the mixture was cooled. Then it was analyzed using the HPLC method. The results of the analysis showed that the concentration of ibotenic acid was below the detection limit, and the concentration of muscimol was 125 μg/ml.

### EXAMPLE 6

Dry fruit bodies of Amanita fungi were sorted to remove off-grade specimens. The selected raw materials, without grinding, were poured with water at a rate of 100 ml per 15 g. The system consisting of liquid and solid material was stirred vigorously for 8 hours. After the extraction was completed, the produced solution was separated from solid particles by filtration under external pressure. A catalyst, which was a compound 2, was added to the filtrate at a rate of 50 g/l. The produced mixture was heated to 75 °C under constant stirring and kept at this temperature for 5 hours. After 5 hours, the mixture was cooled. Then it was analyzed using the HPLC method. The results of the analysis showed that the concentration of ibotenic acid was below the detection limit, and the concentration of muscimol was 115 μg/ml.

### EXAMPLE 7

Dry fruit bodies of Amanita fungi were sorted to remove off-grade specimens. The selected raw materials, without grinding, were poured with water at a rate of 100 ml per 15 g. The system consisting of liquid and solid material was stirred vigorously for 8 hours. After the extraction was completed, the produced solution was separated from solid particles by filtration under external pressure. A catalyst, which was a compound 1, was added to the filtrate at a rate of 50 g/l. The produced mixture was heated to 75 °C under constant stirring and kept at this temperature for 5 hours. After 5 hours, the mixture was cooled. Then it was analyzed using the HPLC method. The results of the analysis showed that the concentration of ibotenic acid was below the detection limit, and the concentration of muscimol was 90 μg/ml.

### EXAMPLE 8

Dry fruit bodies of Amanita fungi were sorted to remove off-grade specimens. The selected raw materials, without grinding, were poured with water at a rate of 100 ml per 15 g. The system consisting of liquid and solid material was stirred vigorously for 8 hours. After the extraction was completed, the produced solution was separated from solid particles by filtration under external pressure. A catalyst, which was a compound 4, was added to the filtrate at a rate of 50 g/l. The produced mixture was heated to 60 °C under constant stirring and kept at this temperature for 4 hours. After 4 hours, the mixture was cooled. Then it was analyzed using the HPLC method. The results of the analysis showed that the concentration of ibotenic acid was below the detection limit, and the concentration of muscimol was 105 μg/ml. The muscimol content in the produced solution according to chromatographic data (206 nm) was 2.2% of the total soluble compounds. The solution was concentrated 50 times under the influence of temperature and reduced pressure. Muscimol in the produced concentrate was purified using the flash chromatography method, with silica gel 60 A as the stationary phase and an aqueous-organic solution of the eluent. Thus, according to chromatographic data (206 nm), the muscimol content was increased to 30% of the total dissolved substances. The produced solution was dried in a vacuum drying oven, after which the dry residue was ground and triturated for homogenization.

### EXAMPLE 9

Dry fruit bodies of Amanita fungi were sorted to remove off-grade specimens. The selected raw materials, without grinding, were poured with water at a rate of 100 ml per 15 g. The system consisting of liquid and solid material was stirred vigorously for 6 hours. After the extraction was completed, the produced solution was separated from solid particles by filtration under external pressure. A catalyst, which was a compound 4, was added to the filtrate at a rate of 50 g/l. The produced mixture was heated to 60 °C under constant stirring and kept at this temperature for 4 hours. After 4 hours, the mixture was cooled. Then it was analyzed using the HPLC method. The results of the analysis showed that the concentration of ibotenic acid was below the detection limit, and the concentration of muscimol was 108 μg/ml. The muscimol content in the produced solution according to chromatographic data (206 nm) was 2.7% of the total soluble compounds. The produced solution was treated using the ion-exchange chromatography method with a strongly and/or weakly acidic cation resin as the stationary phase and several eluents based on aqueous solutions with pH=4-14. Thus, according to chromatographic data (206 nm), the muscimol content was increased to 25% of the total dissolved substances. The produced solution was dried in a lyophilizer, after which the dry residue was ground and triturated for homogenization.

Thus, a method for producing an extract of Amanita genus fungi has been developed, and its implementation ensured the achievement of a technical result consisting in producing an extract with a high yield of muscimol, in which the content of ibotenic acid is minimized.

1. A method for producing an extract of Amanita genus fungi, comprising:
obtaining dried biomass of Amanita genus fungi;
performing aqueous or aqueous-alcoholic extraction of the dried biomass of said fungi to produce an initial extract;
separating solid particles of the biomass of said fungi from the initial extract;
characterized in that, after separating solid particles of the biomass from the initial extract, a decarboxylation reaction of ibotenic acid in said extract is carried out to produce the final extract of Amanita genus fungi by adding to the initial extract, from which solid particles have been separated, a compound of structural formula (III): 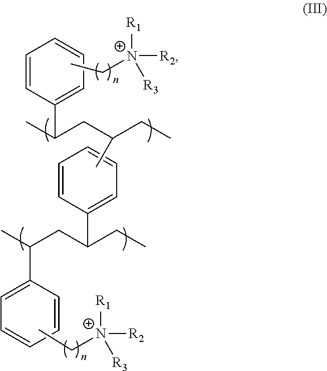
wherein
R₁ is CH₃;
R₂ is selected from the group comprising H and CH₃;
R₃ is selected from the group comprising H, CH₃, (CH₂)ₖCH₃, (CH₂)ₖOH, C₆H₄OH;
k is an integer from 1 to 17;
n is an integer from 1 to 10;
wherein if R₂ is H, R₃ is H,
and if R₂ is CH₃, R₃ is selected from the group comprising CH₃, (CH₂)ₖCH₃, (CH₂)ₖOH, C₆H₄OH.

2. The method of claim 1, characterized in that the aqueous or aqueous-alcoholic extraction of the biomass of Amanita genus fungi is carried out in an inert atmosphere or air atmosphere at normal or reduced pressure.

3. The method of claim 1, characterized in that an organic or inorganic acid in an amount of 0.1-10 wt. % is added to the initial extract after adding the compound of structural formula (III).

4. The method of claim 3, characterized in that the organic acid is selected from the group comprising citric acid, acetic acid, formic acid, sorbic acid.

5. The method of claim 3, characterized in that the inorganic acid is selected from the group comprising hydrochloric acid and/or orthophosphoric acid.

6. The method of claim 1, characterized in that Amanita genus fungi are selected from the group comprising Amanita muscaria, Amanita pantherina, Amanita citrina, Amanita tomentella, Amanita porphyria, Amanita regalis, Amanita albocreata, Amanita aliena, Amanita altipes, Amanita aprica, Amanita armeniaca, Amanita augusta, Amanita breckonii, Amanita chrysoblema, Amanita eliae, Amanita farinosa, Amanita frostiana, Amanita gemmata, Amanita gioiosa, Amanita ibotengutake, Amanita multisquamosa, Amanita nehuta, Amanita orientigemmata, Amanita parcivolvata, Amanita parvipantherina, Amanita persicina, Amanita petalinivolva, Amanita pseudoregalis, Amanita roseotincta, Amanita rubrovolvata, Amanita velatipes, Amanita virgineoides, Amanita viscidolutea, Amanita wellsii, Amanita xanthocephala, Amanita xylinivolva.

7. The method of claim 1, characterized in that either unground or ground biomass is used as the dried biomass of Amanita genus fungi.

8. The method of claim 1, characterized in that the dried biomass of Amanita genus fungi with a residual moisture content of 0.1-20% is used.

9. The method of claim 1, characterized in that the decarboxylation reaction of ibotenic acid in the initial extract is carried out at room temperature or within a temperature range of 30-95 °C for 2-48 hours.

10. The method of claim 1, characterized in that the aqueous or aqueous-alcoholic extraction of the dried biomass is carried out at a temperature of 5-40 °C for 0.5-10 hours.

11. The method of claim 1, characterized in that the compound of formula (III) is solid and insoluble in water and in an aqueous-alcoholic mixture.

12. The method of claim 1, characterized in that, after completing the decarboxylation reaction of ibotenic acid in the initial extract, the compound of structural formula (III) is separated from the final extract by filtration.

13. The method of claim 1, characterized in that the extraction and decarboxylation reaction of ibotenic acid in the initial extract are carried out in a sun-protected container.

14. The method of claim 1, characterized in that the separation of solid particles of the biomass from the initial extract is performed by centrifugation or filtration.

15. The method of claim 1, characterized in that, after completing the decarboxylation reaction of ibotenic acid in the initial extract, the final extract is purified to remove contaminants.

16. The method of claim 1, characterized in that, after completing the decarboxylation reaction of ibotenic acid in the initial extract, the final extract is either concentrated or dried.

17. The method of claim 16, characterized in that concentrating the final extract involves distillation or solvent evaporation.

18. The method of claim 16, characterized in that drying the final extract involves vacuum drying or lyophilization.

19. An extract of Amanita genus fungi produced by a method comprising:
obtaining dried biomass of Amanita genus fungi;
performing aqueous or aqueous-alcoholic extraction of the dried biomass of said fungi to produce an initial extract;
separating solid particles of the biomass of said fungi from the initial extract;
characterized in that, after separating solid particles of the biomass from the initial extract, a decarboxylation reaction of ibotenic acid in said extract is carried out to produce the final extract of Amanita genus fungi by adding to the initial extract, from which solid particles have been separated, a compound of structural formula (III): 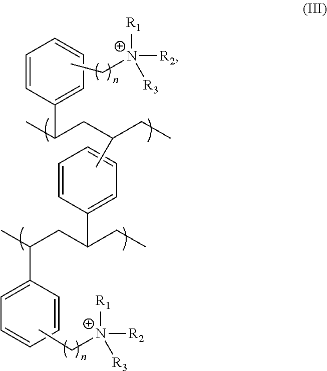
wherein
R₁ is CH₃;
R₂ is selected from the group comprising H and CH₃;
R₃ is selected from the group comprising H, CH₃, (CH₂)ₖCH₃, (CH₂)ₖOH, C₆H₄OH;
k is an integer from 1 to 17;
n is an integer from 1 to 10;
wherein if R₂ is H, R₃ is H,
and if R₂ is CH₃, R₃ is selected from the group comprising CH₃, (CH₂)ₖCH₃, (CH₂)ₖOH, C₆H₄OH.

20. The extract of Amanita genus fungi of claim 19, formulated as a tablet, capsule, powder, pill, granule, solution, suspension, emulsion, tincture, gel, cream, ointment, or chewing gum, for use as a dietary supplement.

21. The extract of Amanita genus fungi of claim 19, formulated as a tablet, capsule, powder, pill, granule, solution, suspension, emulsion, tincture, gel, cream, or ointment, for use as a pharmaceutical composition.
